(19) United States
(12) Patent Application Publication
(10) Pub. No.: US 2022/0199247 A1
(43) Pub. Date: Jun. 23, 2022

(54) TELEMEDICINE STETHOSCOPE DEVICE

(71) Applicant: Sheikh K. Jasimuddin, Orlando, FL (US)

(72) Inventor: Sheikh K. Jasimuddin, Orlando, FL (US)

(21) Appl. No.: 17/128,574

(22) Filed: Dec. 21, 2020

(51) Int. Cl. G16H 40/67

(52) U.S. Cl. CPC G16H 40/67

(57) ABSTRACT

A telemedicine stethoscope system includes a stethoscope device for communicating with a patient interface device, a healthcare provider interface device and a system administration. The stethoscope device includes a main body for housing a sensor suite, and a controller. The sensor suite including a plurality of components for capturing patient information such as heartbeat, pulse oxygen level and temperature, and the controller includes a communication unit for communicating with the patient interface device. The patient interface device includes a user smartphone or tablet, having a telemedicine mobile application with instructions for controlling the operation of the stethoscope device, for conducting an audiovisual telemedicine session with the healthcare provider interface device, and for sending the captured patient information to the healthcare provider interface device during the telemedicine session.

### TECHNICAL FIELD

The present invention relates generally to medical instrumentation, and more particularly to a stethoscope device for use during a telemedicine session.

### BACKGROUND

The statements in this section merely provide background information related to the present disclosure and may not constitute prior art.

Owing to the recent spread and transmission of communicable viruses such as the COVID-19 pandemic of 2020, for example, doctors and governments around the world are asking individuals to reduce travel and to maintain minimum recommended separation distances with other individuals.

To this end, many non-emergency healthcare visits are being conducted remotely whereby the patient and doctor/healthcare provider utilize video chat sessions (e.g., telemedicine). During these sessions, the doctor is able to see and talk to the patient but is unable to utilize any instrumentation to physically examine the patient. As such, the doctor must rely on what the patient reports in order to make a diagnosis and treatment plan.

With the above in mind, it would be beneficial to provide a diagnostic device that can be utilized by a patient under the instruction of a healthcare provider during a telemedicine session to provide real time diagnostic information about the patient to assist the provider in making a diagnosis and treatment plan.

The present invention directed to a telemedicine stethoscope device differs from the conventional art in a number of aspects. The manner by which will become more apparent in the description which follows, particularly when read in conjunction with the accompanying drawings.

### SUMMARY OF THE INVENTION

The present invention is directed to a telemedicine stethoscope system. One embodiment of the present invention can include a stethoscope device for communicating with a patient interface device, a healthcare provider interface device and a system administration. In one embodiment, the stethoscope device can include a main body for housing a sensor suite, and a controller. The sensor suite can include a plurality of components for capturing patient information, and the controller can include a communication unit for communicating with the patient interface device.

In one embodiment, the patient interface device can include a user smartphone or tablet and can include a mobile application having instructions for controlling the operation of the stethoscope device. The patient interface device can also include functionality for conducting an audiovisual telemedicine session with the healthcare provider interface device, and to send the patient information to the healthcare provider interface device.

The system administration can provide the communicative link between the interface devices and can selectively receive and store the patient information.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 shows an exemplary telemedicine stethoscope system that is useful for understanding the inventive concepts disclosed herein.

FIG. 2A is a top perspective view of the stethoscope device in accordance with one embodiment of the invention.

FIG. 2B is a bottom perspective view of the stethoscope device in accordance with one embodiment of the invention.

FIG. 3 is a simplified block diagram of the sensor suite of the stethoscope device, in accordance with one embodiment of the invention.

FIG. 4 is a simplified block diagram of the internal controller of the stethoscope device, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described herein, the term “system administrator” can be used to describe any individual, group or legal entity that is performing the below described system administration 130, in furtherance of the methodology described herein. In one embodiment, the system administrator can be an independent third-party company providing services to healthcare provider for use with their patients. In another embodiment, the system administrator and the healthcare provider can be the same entity.

As described herein, a “healthcare provider” can be any individual, group or legal entity that uses the below described system, device and/or methodology to receive diagnostic information from a patient.

As described herein, “diagnostic information” shall include any/and all data, that is captured, recorded, or derived from or by the below described telemedicine stethoscope.

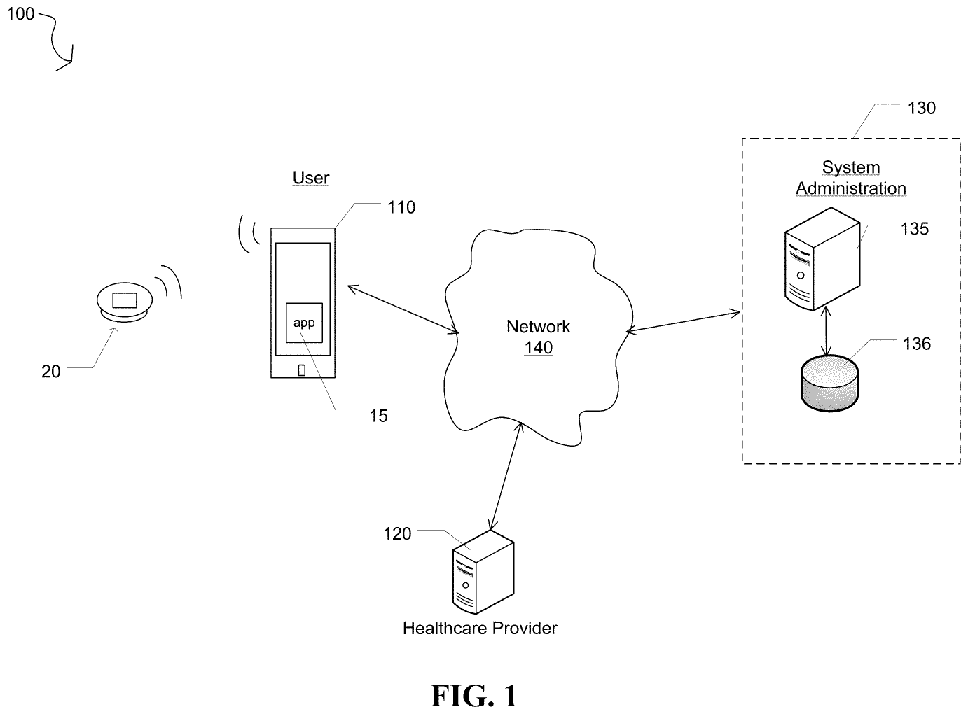

FIG. 1 is a schematic illustration of an exemplary operating environment 100 for utilizing the telemedicine stethoscope device. In one embodiment, the system 100 can include a stethoscope device 20 that is in communication with a patient/user interface device 110, at least one healthcare provider interface device 120, and a system administration 130 that are connected over a network 140.

The network 140 can be any type of network, including a cellular network, local area network (“LAN”), such as an intranet, a wide area network (“WAN”), the internet, and/or any other type of data transmission and reception medium, for example.

In the preferred embodiment, the patient interface device 110 can include a tablet or smartphone device having an onboard camera, a wireless transceiver (e.g., Bluetooth, WiFi), and a telemedicine stethoscope mobile application 15 (e.g., App) for conducting a telemedicine session whereby audio and video information from the smartphone, along with patient information from the stethoscope device 20 is exchanged with the devices 120 and/or 130. In this regard, the App 15 can include functionality for communicating directly with the device 20 to send operating instructions thereto and to receive patient information therefrom.

Of course, the inventive concepts are not limited to this configuration. As such, each of the interface devices 110 and 120 can be any type of computing device that is operable by a human user. A computing device refers to any device with a processor and memory that can execute instructions and communicate with another device. Computing devices include, but are not limited to, smartphones, tablet computers, personal computers, desktop computers, laptop computers and/or purpose-built machines that are encoded with an application interface, so as to perform the functionality so described.

In either instance, each of the interface devices can include one or more client applications, such as a conventional web browser, and/or an application interface, for example, which can allow the device to communicate with other interface devices and/or the system administration 130.

The system administration 130, according to one embodiment, can include one or more individual computing devices 135 that can be connected to one or more databases 136 in which various portions of the below described methodology can be performed. In one embodiment, the system administration 130 can function to provide a central hub for controlling the communication between the interface devices 110 and 120 through any number of different mediums such as a website, a mobile application, or a direct connect audiovisual service such as cellular data, for example. In this regard, one or more of the individual computing devices 135 can include various web servers, email servers, application database servers and so forth.

The database 136 can function to store any type of data, including the system operating instructions for facilitating communication between the device components, routing information and/or generating presentation screens for implementing the below described methodology. To this end, the database can include any type of computer-readable storage mediums, including all forms of volatile and non-volatile memory such as, for example, semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD.

In various embodiments, data captured by the device 20 can be stored on the database 136 and/or may be provided directly to the healthcare provider interface device 120 for storage thereon.

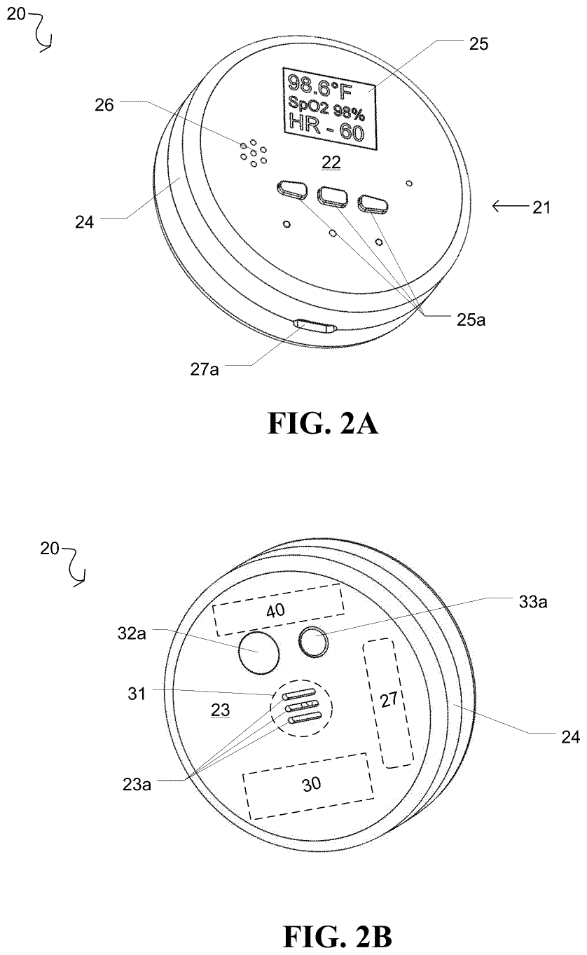

FIGS. 2A-4 illustrate one embodiment of a telemedicine stethoscope device 20 that is useful for understanding the inventive concepts disclosed herein. As shown, the device 20 can include a main body 21, a display unit 25, a speaker 26, a power source 27, a sensor suite 30, and a controller 40.

In the preferred embodiment, each of the device components can be lined with a conductive barrier such as steel or silicone, for example, to prevent signal feedback between the components.

In one embodiment, the main body 21 can include a generally circular shaped member having a top surface 22, a bottom surface 23 and a continuous sidewall 24 that define a watertight interior space. As described herein, the main body 21 may be formed from any number of materials that are, for example, relatively strong and stiff for their weight. Several nonlimiting examples include but are not limited to various lightweight metals or metal alloys (e.g., aluminum, or alloys thereof), or plastic/polymers (e.g., high-density polyethylene (HDPE), rigid polyvinyl chloride (PVC), or polyethylene terephthalate (PET)), for example.

Although described above with regard to a particular shape or size, this is for illustrative purposes only, as the main body may be constructed to comprise any number of different shapes and sizes.

The display unit 25 can be communicatively linked to the below described sensor suite 30 and controller 40 and can include virtually any type of known device capable of presenting information in a digital format to a user via a screen. In one embodiment, the display unit can include an LCD screen having a backlight feature for displaying operating information such as a battery power level, system errors and pairing status with the user interface device 110. Additionally, the screen can display patient information such as heartbeat, pulse oxygen levels, and temperature, for example. As shown, any number of individual buttons 25a can also be provided in order to allow the device 20 to receive operating instructions from a user.

Of course, other embodiments are contemplated wherein the display unit includes a touch screen device capable of providing two-way information with a user via a graphic user interface. Likewise, other embodiments are contemplated wherein the display can include or consist of a series of lights indicating operational status only.

The speaker 26 can be communicatively linked to the below described controller 40 and can include any number of commercially available devices such as a cone, dome or semi-dome tweeter, for example, that can function to generate and/or play an audible sound.

The power source 27 can include one or more batteries capable of providing the necessary power requirements to each element of the device 20. In the preferred embodiment, the batteries can be permanently located within the main body and can be rechargeable in nature via a charging port 27a, such as a mini or micro-USB port, for example. Of course, traditional batteries can also be utilized, and the main body can further include a battery compartment having a removable cover (not illustrated) for allowing a user to access the same.

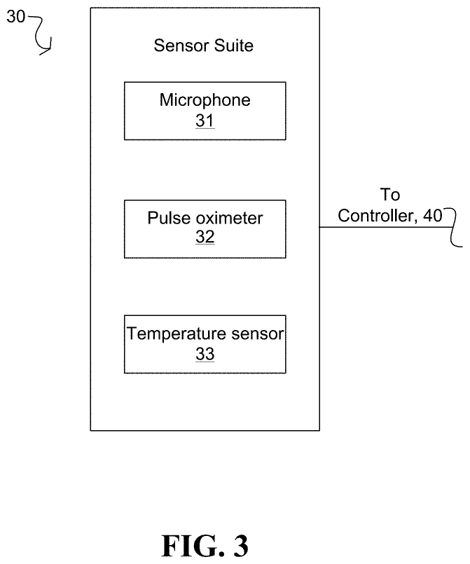

The device 20 can include any number of individual sensors, and/or systems referred to collectively as a sensor suite 30 for capturing patient information. As shown in FIG. 3, one embodiment of a sensor suite 30 for use herein can include a microphone 31, a pulse oximeter 32, and a temperature sensor 33, for example.

The microphone 31 can be communicatively linked to the below described controller 40 and can include any type of sound-oriented input device that can function to detect and capture sounds. In one embodiment, microphone can be positioned above a series of openings 23a that are located along the bottom surface of the main body. The openings using the thickness of the main body material to provide a physical separation distance and an unimpeded path between the microphone's input and a patient's body so as to allow the microphone to capture data such as the patient's heartbeat, for example.

The pulse oximeter 32, be communicatively linked to the below described controller 30 and can include any number of different components capable of detecting and measuring the amount of oxygen in the blood of a patient's body. In one embodiment, the pulse oximeter can include a plurality of Light Emitting Diodes (LEDs) and a photodiode that are positioned behind a lens 32a along the bottom surface 23 of the main body. In operation, the LED's can generate and direct light at different wavelengths, such as 660 nm and 910 nm, for example through the arterial blood of the patient's body. This light can be detected by the photodiode and calculated to determine blood oxygen levels.

Of course, this is but one possible arrangement of components for use in detecting and determining the pulse oxygen level of a patient using the device 20. To this end, any number of other components and arrangement of components are contemplated for use herein.

The temperature sensor 33 be communicatively linked to the below described controller 30 and can include a thermocouple 33a that is positioned along the bottom surface 23 of the main body. The temperature sensor can function to detect the temperature of the patient to which the thermocouple is in contact with, while using the device 20.

Although described above with regard to a contact-type temperature sensor, this is for illustrative purposes only, as any number of other devices such as a thermal temperature sensor, for example, capable of detecting and reporting the temperature of an external object are also contemplated.

The sensor suite 30 can be coupled to the below described control unit 40, so as to receive operating instructions and to allow the patient information to be stored and transmitted to an external device such as the user interface device 110, the healthcare provider interface 120 and/or the system administration 130 in real time. Although described above as including specific components 31-33, this is for illustrative purposes only, as those of skill in the art will recognize that any number of additional sensors/components can be provided in order to capture any type of patient information.

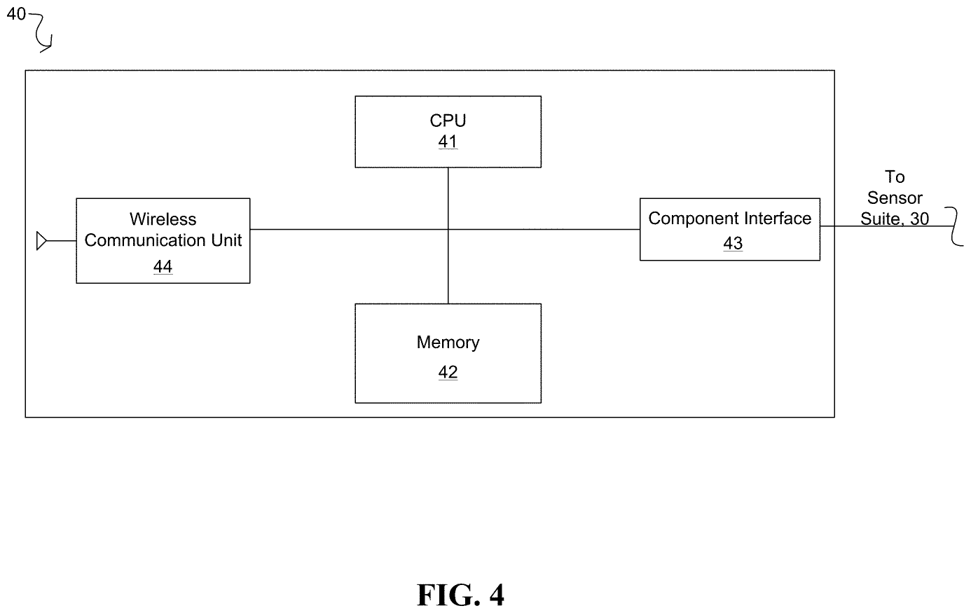

FIG. 4 is a simplistic block diagram illustrating one embodiment of the controller 40, which can control an operation of the sensor suite 30 and can store/transmit the patient information for real time analysis. As shown, the controller can include a processing unit 41 that is conventionally connected to an internal memory 42, a component interface unit 43, and a wireless communication unit 44.

Although illustrated as separate elements, those of skill in the art will recognize that one or more system components 31-33 and/or 41-44 may comprise or include one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 41 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 41 to execute. Although illustrated in FIG. 4 as a single component, memory 42 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a solid-state hard drive, for example.

The bulk storage device can contain any number of different programs that permit the processor to perform the functionality described herein, such as controlling the operation of each element of the sensor suite 30, and for storing the patient information received therefrom, for example.

The component interface unit 43 can function to provide a communicative link between the processing unit 41 and various system elements such as the display unit 25, the speaker 26, the individual sensors of the sensor suite 30, and the communication unit 44, for example. In this regard, the component interface unit can include any number of different components such as one or more PIC microcontrollers, standard bus, internal bus, connection cables, and/or associated hardware such as USB cables and connectors, and other such hardware capable of linking the various components. Of course, any other means for providing the two-way communication between the system components can also be utilized herein.

The communication unit 44 can include any number of components capable of sending and/or receiving electronic signals with another device, either directly or over a network. In one preferred embodiment, the communication unit 44 can include a Bluetooth transceiver having an antenna 44a for communicating wirelessly with an external device such as the smartphone/user interface device 110 illustrated at FIG. 1, for example. Such a feature allowing the patient information captured by the sensor suite to be sent directly to the user device 110 for transmission to a healthcare provider during a telemedicine session in real time.

Of course, the communication unit is not limited to the use of Bluetooth communication, as any number of other transmission and reception mechanisms and protocols can also be utilized herein. Several nonlimiting examples include WiFi, and Near-Field-Communication (NFC) devices, for example. Likewise, communication over a cable can also be performed using the above-described micro-USB port 27a, for example.

Although described above as providing direct communication between the device 20 and user interface 110, other embodiments are contemplated wherein the communication unit 44 of the device 20 can communicate directly with the healthcare provider interface 120 and/or the system administrator 130 in addition to the user interface 120 or instead of the user interface 120. In such situations, the communication unit 44 can include a cellular transceiver or an internet enabled transceiver for communicating over a wireless network, for example.

In operation, a patient can use their user interface device 110 to establish a video conference session with a healthcare provider who is using their own interface device 120. When connected, the provider can instruct the patient to pair the stethoscope device 20 with their device 110 (e.g., Bluetooth) using the display screen and buttons. Once paired, the healthcare provider can instruct the patient when and where to place the stethoscope on their body in order to capture patient information.

The ability of the healthcare provider to use the camera of the patient interface device to watch the patient physically place the stethoscope device at the instructed location is an important feature of the inventive concepts, as this allows the healthcare provider to be sure the device is being operated correctly to capture the vital diagnostic information from the patient.

In one embodiment, the patient information captured by the stethoscope device 20 can be transmitted directly to the user device 110 which can then forward the information to the healthcare provider via the established telemedicine video session.

In another embodiment, the patient information captured by the stethoscope device 20 can be transmitted to the system administrator for storage or can be routed through the system administrator before reaching the healthcare interface device. Such an embodiment being consistent with a subscription program permitting use of the inventive system.

In yet another embodiment, the healthcare provider can remotely operate the sensor suite of the stethoscope device by sending commands to the patient interface device which are then automatically sent to the stethoscope device. Such a feature advantageously allowing the healthcare provider to be in complete control of the device 20 when examining the patient.

In either instance, the above-described device, system and methodology provide a novel and innovative way for allowing healthcare providers to receive real time diagnostic data about a patient during a telemedicine visit.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1. A telemedicine stethoscope device, comprising:
a main body having a top surface, a bottom surface and an interior space;
a sensor suite positioned within the main body, said sensor suite functioning to capture patient information; and
a controller that is positioned within the main body, said controller including a communication unit that is configured to send the captured patient information to an external device in real time.

2. The device of claim 1, wherein the patient information includes a heartbeat of a patient on which the main body is positioned.

3. The device of claim 1, wherein the patient information includes a blood oxygen level of a patient on which the main body is positioned.

4. The device of claim 1, wherein the patient information includes a temperature of a patient on which the main body is positioned.

5. The device of claim 1, wherein the sensor suite includes a microphone that is configured to detect a heartbeat of a patient on which the main body is positioned.

6. The device of claim 1, wherein the sensor suite includes a pulse oximeter that is configured to detect a and measure a blood oxygen level of a patient on which the main body is positioned.

7. The device of claim 1, wherein the sensor suite includes a temperature sensor that is configured to detect a temperature of a patient on which the main body is positioned.

8. The device of claim 1, further comprising:
a display that is positioned along the main body, said display functioning to independently display the captured patient information.

9. The device of claim 1, wherein the controller includes:
a memory for storing the captured patient information; and
a processor.

10. The device of claim 1, further comprising:
a telemedicine stethoscope application that includes machine readable instructions for execution on a computing device having a processor, a memory, and a display screen, said application functioning to communicate directly with the communication unit of the controller.

11. A telemedicine stethoscope system, comprising:
a stethoscope device having a main body that includes a top surface, a bottom surface and an interior space, a sensor suite positioned within the main body, said sensor suite functioning to capture patient information; and a controller having a communication unit that is positioned within the main body;
a patient interface device;
a healthcare provider interface device; and
a system administration that is in communication with each of the patient interface device, and the health care provider interface device,
wherein the stethoscope device includes functionality for transmitting the captured patient information to the patient interface device.

12. The system of claim 11, wherein each of the patient interface device and the health care provider interface device include functionality for conducting an audiovisual telemedicine session.

13. The system of claim 12, wherein the patient interface device includes functionality for transmitting the captured patient information to the healthcare provider interface device during the audiovisual telemedicine session.

14. The device of claim 12, wherein the patient information includes a heartbeat of a patient on which the main body is positioned.

15. The device of claim 12, wherein the patient information includes a blood oxygen level of a patient on which the main body is positioned.

16. The device of claim 12, wherein the patient information includes a temperature of a patient on which the main body is positioned.

17. The device of claim 11, wherein the sensor suite includes a microphone that is configured to detect a heartbeat of a patient on which the main body is positioned.

18. The device of claim 11, wherein the sensor suite includes a pulse oximeter that is configured to detect a and measure a blood oxygen level of a patient on which the main body is positioned.

19. The device of claim 11, wherein the sensor suite includes a temperature sensor that is configured to detect a temperature of a patient on which the main body is positioned.
